(19) United States
(12) Patent Application Publication
(10) Pub. No.: US 2018/0147262 A1
(43) Pub. Date: May 31, 2018

(54) NOVEL SKIN MEDICAL AND COSMETIC CARE PRODUCT

(71) Applicant: University of Bremen, Bremen (DE)

(72) Inventor: Ursula MIRASTSCHIJSKI, Bremen (DE)

(21) Appl. No.: 15/569,925

(22) PCT Filed: May 2, 2016
(86) PCT No.: PCT/EP2016/059789

§ 371 Date: Oct. 27, 2017

(30) Foreign Application Priority Data
Apr. 30, 2015 (EP) 15166070.1

(51) Int. Cl. A61K 38/17; A61K 9/00; A61K 45/06; A61P 17/02; A61P 37/08; A61K 8/64; A61Q 19/08; A61Q 7/00

(52) U.S. Cl. CPC A61K 38/395; A61K 9/0014; A61K 45/06; A61Q 7/00; A61P 37/08; A61K 8/64; A61Q 19/08; A61P 17/02

(57) ABSTRACT

Provided are novel skin medical and cosmetic care products as well as compositions for the treatment of the skin based on pulmonary surfactants.

### FIELD OF THE INVENTION

The present invention generally relates to skin care and treatment products based on a pulmonary surfactant or a derivative thereof. In particular, the present invention relates to compositions comprising a pulmonary surfactant or a biologically active derivative thereof for use in prevention or treatment of skin injuries and/or disorders. Furthermore, the present invention relates to a use of such compositions for dermatological and cosmetic treatments of the skin. In addition, the present invention relates to pharmaceutical compositions, packages and products designed for the application to, preferably onto the skin comprising said compositions comprising a pulmonary surfactant or a biologically active derivative thereof.

### BACKGROUND OF THE INVENTION

In Western European countries, the incidence of thermal injuries is between 4%¹ and 10%.² In Germany, 20.000 burn injuries are registered per year with 4.000 patients requiring general hospital care and 1.200 being admitted to specialized intensive care units of burn centers.¹ Children and elderly people are high risk groups for burn injuries with incidences between 15%³ and 58%⁴ or 20%², respectively. In deep and full-thickness burns, necrotic tissue is surgically removed and commonly replaced with split-thickness skin grafts. After completion of wound healing, continuous aftercare is required with ointments, physiotherapy, occupational therapy, and silicone sheeting to prevent hypertrophic scar formation. In addition, individually tailored pressure garments are prescribed although evidence for its beneficial effects is low.⁵⁻⁷ Deep burns lead to scarring and contractures. The incidence of pathologic scarring after burn injury varies between 30% and 91%^{6,8} with wound depth and total body surface area burned being predictors for severe scarring.⁹ In case of mutilating scarring, secondary surgical interventions are required with scar release and plastic-surgical reconstruction. Approximately 0.3 billion EUR in total or 270.000 EUR per patient/year were spent on burn sequelae¹⁰. Treatment of scars causes higher costs than for other burn sequelae because of multiple surgical interventions for surgical interventions due to scarring¹¹.

### Physiology of Skin Wound Healing

Main goal for wound repair is the restoration of an intact barrier against physical, chemical and microbial damage¹². Reepithelialisation at the air-liquid-interphase is paralleled by wound surface minimization. After accomplished wound closure, keratinocytes commit to terminal differentiation and form a new multilayered epidermis. This change of morphology leads to a shrinkage of the cell's basal contact area with concomitant remodelling of the extracellular environment, e.g. formation of hemidesmosomes, desmosomes and adherence junctions with tight intercellular contacts¹³. Thereby, epidermal cells contract and minimize effectively their outer surface at the air-liquid interphase. Differences in mechanical tension are transmitted to the underlying extracellular matrix and stimulate along with secreted growth factors myofibroblast alignment and contraction¹⁴. Alpha-smooth muscle actin (ASMA) containing myofibroblasts play an important role in the second phase of wound contraction¹⁴. Mechanistically, intracellular actin microfilaments terminate in adhesion complexes at the cell surface connecting intracellular actin bundles with the extracellular matrix (ECM). Thereby mechanical force is generated by myofibroblasts and transmitted into the surrounding ECM resulting in matrix reorganization and wound contraction ¹⁴.

### Pathophysiology of Aberrant Wound Healing

Aberrant skin wound healing is characterized by two diametrically opposed entities: chronic, non-healing wounds and excessive wound healing with scarring and contracture formation.

Chronic or non-healing wounds are open wounds that fail to epithelialize and close in a reasonable amount of time, usually defined as 30 days. These wounds typically are clinically stagnant and unable to form robust granulation tissue. Many factors contribute to inhibit healing in these patients, but no unifying theory can explain the etiopathogenesis of each individual non-healing wound.¹⁵ Many medical conditions contribute to impaired wound healing, e.g., diabetes, arterial insufficiency, venous disease, lymphedema, pressure necrosis, infection, and many more.¹⁵ Studies have implicated an altered expression profile of growth factors in diabetic wounds.¹⁶ Sustained inflammation is marked by an increase in proinflammatory cytokines¹⁷ and cells, e.g., macrophages, B-cells, plasma cells in the wound margin.¹⁸ MMP production is deregulated with dislocation of proinflammatory MMP-9 to the ulcer bed.^{19,20} Advanced glycation end-products and inflammatory mediators commit fibroblasts and vascular cells to apoptosis and impair granulation tissue formation.²¹ Diabetic fibroblasts and keratinocytes have reduced proliferation rates and collagen production.²²

### State-of-the-Art Treatment of Non-Healing Wounds

In each case, treatment begins with debridement of any necrotic tissue present.²³ However, despite optimal treatment for each clinical problem, these wounds frequently still do not heal and surgical intervention is required. Treatment begins with debridement of necrotic tissue, which removes a potential source of bacterial infection. Depending on the bacterial load, systemic antimicrobial treatment adjusted to bacterial drug resistance is recommended. To provide optimal healing conditions, wound moisture imbalances should be corrected with adequate dressing and compression therapy.²⁴ Active medical co-morbidities are aggressively treated. Care is continued until the wound is clean and ready for reconstruction or heals by secondary intention that can proceed for several months.

### Pathophysiology of Excessive Scarring and Fibrosis

Normal wounds have “stop” signals that halt the repair process when the dermal defect is closed and epithelialization is complete. When these signals are absent or ineffective, the repair process may continue unabated and cause excessive scarring. A lack of programmed cell death, apoptosis, at the conclusion of repair with continued presence of activated fibroblasts, e.g., myofibroblasts, secreting extracellular matrix (ECM) components has been implicated.²⁵ Hypertrophic scar fibroblasts produce more connective tissue growth factor (CTGF) after TGF-β stimulation.²⁶ CTGF stimulate chemotaxis, proliferation, matrix metalloproteinase (MMP) expression and ECM production.²⁷ The proteoglycan decorin binds TGF-β and regulates collagen fibrillogenesis by downregulating TGF-β production.^{28,29} In contrast to normal dermal fibroblasts, hypertrophic scar-derived fibroblasts secrete less decorin and probably contribute thereby to sustained TGF-β activity.³⁰ Programmed cell death is thought to underlie myofibroblast disappearance after wound closure with persisting myofibroblasts in excessive scarring.¹⁵

### State-of-the-Art Treatment for Scars

Patients who are at increased risk for excessive scarring benefit from preventive techniques, which include silicone gel sheeting or ointments, hypoallergenic microporous tape, and concurrent intralesional steroid injection.³¹ Despite preventive measures, excessive scarring may occur and lead to scar contractures. Multimodality therapies are generally used to treat excessive scarring; these include silicone gel sheeting, custom-fitted pressure garments, and physical therapy alone or with massage, electrical stimulation, or ultrasound. Steroid injection of especially difficult areas is sometime necessary. Laser treatment can be useful.³² Surgical treatment with Z-plasty, excision and grafting, and flap reconstruction is frequently required.³¹ Until now there are no therapies that promote skin wound healing, attenuate concomitant inflammatory reaction and prevent effectively excessive scarring. Accordingly, there is a need for new treatment methods and substances for use in treatment of wounds, local inflammation and scar formation with improved qualities in concern of these treatment aims.

This technical problem has been solved by the embodiments of the present invention as characterized in the claims and described further below.

### SUMMARY OF THE INVENTION

The present invention generally relates to skin medical and care products including pharmaceutical compositions based on a pulmonary surfactant, which are particularly suited for the topical treatment of skin injuries while avoiding or preventing excessive scarring and other aberrations of the skin such as fibrosis often associated with wound closure.

Pulmonary, i.e. lung surfactants are complex compositions of lipids and proteins which are produced by the alveolar cells in the lungs and cover the alveolar air-liquid interface. They facilitate recruitment of collapsed airways, increase pulmonary compliance, i.e. the ability of the lungs and the thorax to expand, and prevent end-expiratory alveolar collapse probably by reducing the surface tension and viscosity.⁴² Furthermore, they reduce fluid accumulation and are involved in the innate immune response in the lung.⁴³

Hitherto, pulmonary surfactants have been applied in the treatment of disorders related to surfactant deficiency which is the major factor leading to respiratory distress syndrome of the newborn (IRDS) and to adult respiratory distress syndrome (ARDS), wherein the surfactant is administered by inhalation or aspiration to the patients. Pulmonary surfactants for use in treatment of respiratory distress syndromes are described, for example, in the international application WO 2011/029525.

The present invention is based on the surprising observation that a modified natural pulmonary surfactant, i.e. bovactant (Alveofact©) is capable of promoting epithelial migration of keratinocytes in a cell based assay and enhancing wound closure in a pertinent standardized excisional wound healing model in mice, wherein the treatment is accompanied with a reduction of (pro-)inflammatory cytokines and reduce scar formation. Without intending to be bound by theory, in accordance with the experiments described in the appended Examples it is believed that the effect of lung surfactant on wound resurfacing and contraction is because of epidermal keratinocytes at the air-liquid-interphase trigger wound contraction by minimization of the wound surface. Thereby they induce matrix contraction by myofibroblasts in the dermal compartment. Locally applied surfactant-like substances could reduce surface tension and influence wound healing with its antimicrobial surfactant proteins. Indeed, the findings of the present invention translate into clinical use of pulmonary surfactants for skin wound healing with special emphasis on treatment of burn wounds. This novel approach marks a turnaround in skin wound therapy with fundamental impact not only for wound healing research but also for patients' lives.

Accordingly, the findings obtained in the experiments performed within the scope of the present invention provide a novel use of pulmonary surfactants and biologically active derivatives thereof in the treatment of a broad variety of skin disorders, including but not restricted to skin wounds, fibrosis, burns, tissue augmentation, tissue defects, inflammation, irritations, allergies, benign or malignant malformations, scar formation and complementary treatment in combination with skin grafts, reconstructive surgery and dermatosurgery or any topical, intra-epidermal or intra-cutaneous skin treatment as well as in non-medical treatments such as dermatological or cosmetic treatment of scars, wrinkles, dyscolorations of the skin, skin irritations, volume augmentation, baldness, treatments after skin peeling, dermabrasio, medical needling and also in any topical, intra-epidermal, intra-cutaneous or subcutaenous skin treatments.

Furthermore, the present invention also relates to a use of a pulmonary surfactant or a biologically active derivative thereof for promoting epithelial migration of keratinocytes in vitro or in vivo.

### Definitions

“Unit dose” refers to the amount of the surfactant administered to a patient in a single dose. The unit dose can be calculated in relation to the body weight and/or skin type.

The term “about” as used herein defines a possible deviation from the concerned value in the range of 1%-30%, in particular of 1%-20%, in particular of 1%-10%, in particular of 1%-5%, in particular of 1%-2% of the value as defined without usage of this term.

If not explicitly specified otherwise, the term “pulmonary surfactant” as used herein refers to natural surfactants recovered, e.g., from lungs or amniotic fluid⁴⁶; modified natural surfactants extracted, e.g., from lungs or amniotic fluid as well and preferably supplemented with lipids and/or surfactant proteins or other surface active material; artificial; and reconstituted surfactants. According to Wilson, Expert Opin. Pharmacother. 2 (2001), 1479-1493, these classes of pulmonary surfactants can be specified as follows:

(i) “natural” surfactants which are those recovered intact from lungs or amniotic fluid without extraction and have the lipid and protein composition of natural, endogenous, surfactant.

(ii) “modified natural” surfactants which are lipid extracts of minced mammalian lung, lung lavage or amniotic fluid. Due to the lipid extraction process used in the manufacture process, the hydrophilic proteins SP-A and SP-D are lost or greatly reduced in amount. These preparations have variable amounts of SP-B and SP-C and, depending on the method of extraction, may contain non-surfactant lipids, proteins or other components. Some of the modified natural surfactants present on the market, like Survanta (vide ultra) are spiked with synthetic components such as tripalmitin, dipalmitoylphosphatidylcholine and palmitic acid;

(iii) “artificial” surfactants which are simply mixtures of synthetic compounds, primarily phospholipids and other lipids that are formulated to mimic the lipid composition and behaviour of natural surfactant. They are devoid of surfactant apoproteins;

iv) “reconstituted” surfactants which are artificial surfactants to which have been added surfactant proteins/peptides isolated from animals or proteins/peptides manufactured through recombinant technology such as those described in international application WO 95/32992, or synthetic surfactant protein analogs such as those described in international applications WO 89/06657, WO 92/22315 and WO00/47623.

“Pharmaceutical acceptable” refers to a medium that do no not produce an allergic or similar untoward reaction when administered to an infant.

“Surfactant activity” for a pulmonary surfactant preparation is defined as the ability to lower the surface tension. The in vitro efficacy of exogenous surfactant preparations is commonly tested by measuring its capability of lowering the surface tension using suitable apparatus such as Wilhelmy Balance, Pulsating Bubble Surfactometer, Captive Bubble Surfactometer and Capillary Surfactometer.

Hitherto, the in vivo efficacy of exogenous surfactant preparations is tested by measuring lung mechanics in pre-term animal models according to known methods. In accordance with the present invention the in vivo efficacy of the pulmonary surfactant preparation and composition comprising the same, e.g. capability of enhancing wound closure, anti-inflammatory effect and/or pro-migratory effect can be tested using the cell-based assay and animal model described in the Examples.

A derivative of an pulmonary surfactant, for example artificial and reconstituted pulmonary surfactant is said to be biologically active in accordance with the present invention if it displays the essential features of the modified pulmonary surfactant bovactant illustrated in the Examples, i.e. being capable in a dose-dependent manner of promoting epithelial migration of keratinocytes in monocultures using a scratch wounding model while fibroblast migration and contractility is not substantially affected, and/or being capable of excisional wound healing in the animal model. In addition, or alternatively, in order to assess the suitability of derivatives of pulmonary surfactants for the purpose of the present invention expression profiling as illustrated in Examples 2 and 3 can be performed, wherein the derivative preferably displays a similar or substantially identical expression pattern like bovactant, i.e. reducing the expression of Tumor Necrosis Factor alpha (TNF-alpha) including downstream signaling molecules and proteins or processes induced by TNF-alpha signaling, TNF-alpha converting enzyme (TACE) and/or other pro-inflammatory cytokines or proteases or mediators, and reducing TNF-alpha receptors; and/or anti-fibrotic, e.g., reducing myofibroblasts or the differentiation of cells into myofibroblasts, or the activation of pro-fibrotic cells or other cells secondarily inducing fibrosis, e.g., reducing transforming growth factor-beta (TGF-beta) including downstream signaling, TGF-beta receptors, angiotensinogen, angiotensin, angiotensinII-receptors (ATII-R) and downstream signaling molecules and/or processes induced by ATII and angiotensin receptors, and/or connective tissue growth factor (CTGF) and downstream signaling molecules or processes induced by all named molecules.

Means and methods for preparing derivatives of pulmonary surfactants such as of the modified natural surfactants poractant alfa, calfactant, bovactant, or beractant are known to the person skilled in the art; see, e.g. international application WO 02/17878, at page 27 in table 1 (taken from D. Gommers, Thesis 1998 at the University of Rotterdam, “Factors affecting surfactant responsiveness”), in Rüdiger et al. (2005)⁴², e.g. in table 1 at page L380; Herting et al. (2001)⁴⁴, e.g., in section Surfactant at page 45 and in FIG. 1 at page 46; and Bernhard et al. (2000)⁴⁵, e.g., in section “Commercially available therapeutic surfactants” and citations 15 and 16 therein, the content of which is herewith incorporated by reference in its entirety.

### BRIEF DESCRIPTION OF THE FIGURES

FIG. 1: Keratinocyte migration with or without Alveofact at different concentrations. Human primary keratinocytes were cultured until confluence and migration induced by a standardized scratch and monitored over 3 days. Alveofact at 0.01 mg/ml (dotted line with triangles) increased epithelial migration compared to control (line with two dots in gaps and rhombs), or Alveofact at 0.1 mg/ml (line with gaps and squares) or 1 mg/ml (continuous line with circles). Mean±SD.

FIG. 2: Primary human fibroblasts were seeded into collagen gels and gel contraction was monitored over 7 days. (A) Alveofact in saline or saline alone (control) was added to incubation media. Alveofact did not influence fibroblast migration (not shown) or contractility.(B) Macroscopic appearance of experimental model with fibroblast seeded collagen gels at different time points.

FIG. 3: Macroscopic wound closure (A) and microscopic wound length (B) of excisional wounds after 14 days of treatment with either control (NaCl=saline), fatty gauze, 0.01 or 0.5 mg/ml Alveofact. Mean±SD.

FIG. 4: (A) Epidermal width assessed by light microscopy of HE stained (hematoxylin and eosin) sections. Mean±SD. Light microscopic pictures of (B): normal skin, (C)-Alv/NaCl-treated skin and (D)-fatty gauze treated skin.

FIG. 5: Gelatin zymography of homogenized wound tissues incubated over 18 h. No differences in active and latent MMP-2 bands were found between groups. Reduced levels of MMP-9 are noted with Alv 0.01 treatment. Standards of recombinant mouse MMP-2 and rmMMP-9 were run in parallel. Arrows depicting bands for MMP-2 and MMP-9.

FIG. 6: Western immunoblotting of excised wounds for ASMA (Alpha-smooth muscle actin) after treatment with NaCl (control) or 0.01 or 0.5 mg/ml Alveofact for 8 days. Note reduced levels of ASMA with Alv 0.01. MW molecular weight marker. Standards of recombinant mouse MMP-3 and recombinant human rhMMP-10 were run in parallel.

FIG. 7: Western immunoblotting for E-cadherin of excised wounds after treatment with NaCl (control) or 0.01 or 0.5 mg/ml Alveofact for 8 days. Note reduced levels of fragmented and full-size E-cadherin with Alveofact 0.01. MW molecular weight marker. Arrows right at the figure depict E-cadherin at 24 kDa and approximately 60 kDa.⁴¹

### DETAILED DESCRIPTION OF THE INVENTION

The present invention generally relates to skin care and treatment products including pharmaceutical and cosmetic compositions based on a pulmonary surfactant, which are adapted and/or designed for the application to, preferably onto the skin, and which are useful in the treatment of acute and chronic skin wounds, and for the prevention and treatment of scarring and fibrosis.

In a first aspect, the present invention relates to a composition comprising a pulmonary surfactant or a biologically active derivative thereof for therapeutic use in the prevention or treatment of a disorder of the skin.

Disorders of the skin include but are not limited to wounds, irritations or chirurgical treatments affecting the normal coherence and/or function of the skin A skin wound is typically understood as a breach in the continuity of skin tissue that is caused by direct injury to the skin such as “lesion”, “cut”, “excoriation”. Several classes generally characterize skin wounds, e.g. punctures, incisions, including those produced by a variety of surgical procedures, excisions, lacerations, abrasions, atrophic skin or necrotic wounds and burns, including large burn areas. In a preferred embodiment, the composition for use according to the present invention is provided, wherein the disorder is selected from the group consisting of skin wounds, fibrosis, burns, tissue augmentation, tissue defects, inflammation, irritation, allergy, inherited or acquired skin diseases with concomitant inflammation, benign or malignant malformations, scar formation and complementary treatment in combination with skin grafts, reconstructive surgery and dermatosurgery or any topical, intra-epidermal, intra-cutaneous or subcutaneous skin treatment.

In a further aspect, the present invention relates to a composition comprising a pulmonary surfactant or a biologically active derivative thereof for non-medical treatment of the skin, e.g. skin conditions which are not pathological but associated with undesired dermatological skin aberrations. Accordingly, the present invention also relates to the use of a composition comprising a pulmonary surfactant or a biologically active derivative thereof for a non-medical, e.g., cosmetic treatment of the skin, for example, for the treatment of scars, wrinkles, dyscolorations, of the skin, skin irritation, volume augmentation, baldness, after skin peeling, dermabrasio and medical needling,

In a preferred embodiment, modified natural pulmonary surfactants are used in accordance with the present invention such as calfactant (Infasurf®, ONY, Inc. Amherst, N.Y., USA), bovactant (Alveofact®, Boehringer Ingelheim Pharma, Ingelheim, Germany), poractant alfa (Curosurf®, Chiesi Farmaceutici SpA, Parma, Italy) and beractant (Survanta®, Abbvie Inc., North Chicago, Ill., USA). Bovactant is obtained by lipid extraction from bovine lung lavage, beractant is prepared by lipid extraction of minced bovine lungs, poractant alfa is an extract of natural porcine lung surfactant and calfactant is derived from calf lungs.

Examples of synthetic/artificial surfactants are lucinactant (Surfaxin®), Colfosceril palmitate (Exosurf®) and Lusupultide (Venticute®). All the synthetic/artificial surfactants comprise mainly the surfactant lipid components and have a greatly reduced protein content or do not comprise proteins or peptides at all. In order to regain some of the functions of the surfactant proteins, Lucinactant comprises peptide fragments mimicking the repeating pattern of hydrophobic and hydrophilic residues in C-terminus of SP-B, and lusupultide comprises recombinant SP-C. Colfosceril palmitate is a protein-free surfactant preparation, it contains DPPC, hexadecanol, and tyloxapol in a relation of 13.5:1.5:1, with 84% DPPC in relation to mass as the only phospholipide, wherein hexadecanol and tyloxapol mimic, to some degree, the functions of the surfactant proteins.⁴⁵

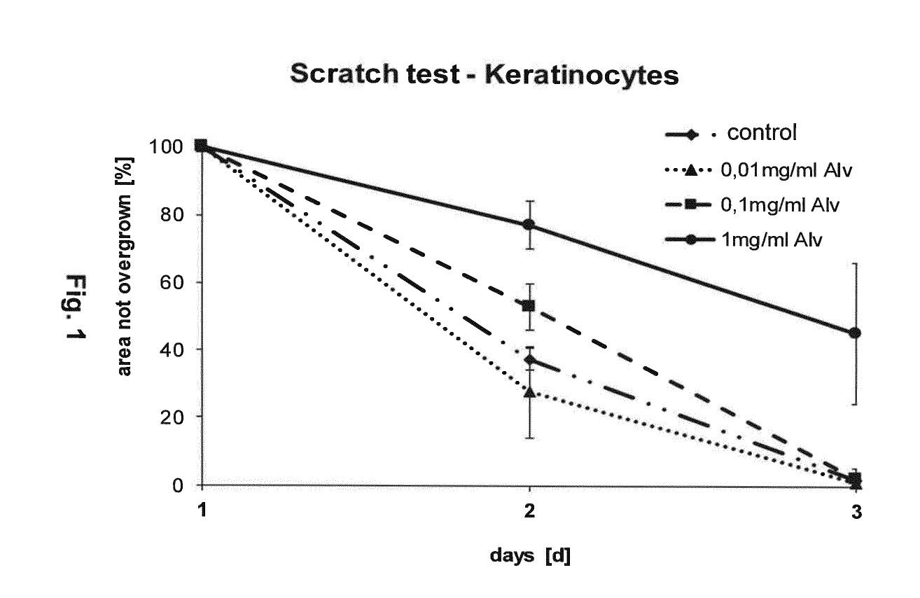

As mentioned, in the modified natural pulmonary surfactants the amount of surfactant proteins is greatly reduced to about 1%, wherein SP-B and SP-C are still associated with the phospholipids but the amounts of SP-A and SP-D are greatly reduced or even not detectable.⁴⁵ The compositions of the different surfactants are described, for example, in international application WO 02/17878 at page 27 in table 1 (taken from D. Gommers, Thesis 1998 at the University of Rotterdam, “Factors affecting surfactant responsiveness”), in Rüdiger et al. (2005)⁴², e.g. in table 1 at page L380; Hefting et al. (2001)⁴⁴, e.g., in section Surfactant at page 45 and in FIG. 1 at page 46; and Bernhard et al. (2000)⁴⁵, e.g., in section “Commercially available therapeutic surfactants” and citations 15 and 16 therein, the disclosure content of which is herewith incorporated by reference in its entirety.

Preferably, the compositions of the present invention comprise a modified pulmonary surfactant or biologically active derivative thereof that has a similar composition as natural surfactants, i.e. comprising lipid and protein components. In particular, as mentioned above, contrary to the modified pulmonary surfactants used in the prior art such as in US patent application US 2010/0048514, which are based on the phospholipid component of the lung surfactant, the pulmonary surfactant used in accordance with the present invention preferably and advantageously comprises at least hydrophilic protein SP-A, preferably in addition SP-B and SP-C, and most preferably in addition or alternatively SP-D. In this context, as described above any of the natural hydrophilic proteins may be substituted by protein fragments, equivalent proteins or peptides mimicking pattern of hydrophobic and hydrophilic residues in (part) of the natural proteins and exhibiting, in context with entire preparation of the pulmonary surfactant the same biological activity; see supra. In a preferred embodiment of the present invention, the pulmonary surfactant is selected from the group consisting of poractant alfa, calfactant, bovactant, and beractant. Most preferably, the modified pulmonary surfactant is bovactant.

In order to have the desired therapeutic or cosmetic effect the composition of the present invention is preferably contacted with the skin area to be treated. Accordingly, in one embodiment of the present invention the composition is designed for topical, intralesional, intraepithelial, intra-epidermal, intra-cutaneous or subcutaneous administration into or preferably onto the skin. In a preferred embodiment, the composition of the present invention is designed for topical administration onto the skin, e.g. wounded skin.

Preferably, the pulmonary surfactant or biologically active derivative thereof in the composition for use according to the present invention is formulated with a pharmaceutical acceptable carrier. Pharmaceutically acceptable carriers and administration routes can be taken from corresponding literature known to the person skilled in the art. The pharmaceutical compositions of the present invention can be formulated according to methods well known in the art; see for example Remington: The Science and Practice of Pharmacy (2000) by the University of Sciences in Philadelphia, ISBN 0-683-306472, Vaccine Protocols. 2nd Edition by Robinson et al., Humana Press, Totowa, N.J., USA, 2003; Banga, Therapeutic Peptides and Proteins: Formulation, Processing, and Delivery Systems. 2nd Edition by Taylor and Francis. (2006), ISBN: 0-8493-1630-8. Examples of suitable pharmaceutical carriers are well known in the art and include phosphate buffered saline solutions, water, emulsions, such as oil/water emulsions, various types of wetting agents, sterile solutions etc. Preferably, the pharmaceutical carrier is a suitable physiologically tolerable solvent, preferably an aqueous, amphiphilic or lipophilic solvent. In a preferred embodiment, the solvent is an aqueous solution such as a sodium chloride solution, Ringer solution or Ringer-acetate solution, preferably sterile, which may also comprise pH buffering agents and other pharmaceutically acceptable excipients such as polysorbate 20, polysorbate 80 or sorbitan monolaurate as wetting agents and sodium chloride as isotonicity agent, preferably at a concentration 0.9% w/v. Compositions comprising such carriers can be formulated by well-known conventional methods. These pharmaceutical compositions can be administered to the subject at a suitable dose. Administration of the suitable compositions may be effected by different ways. As indicated above, preferably the administration is performed by topical, intralesional, intraepithelial, intra-epidermal, intra-cutaneous or subcutaneous methods on or into the skin methods. Aerosol formulations such as wound spray formulations may include purified aqueous or other solutions of the active agent with preservative agents and isotonic agents. Formulations for rectal or vaginal administration may be presented as a suppository with a suitable carrier; see also O'Hagan et al., Nature Reviews, Drug Discovery 2(9) (2003), 727- 735. Further guidance regarding formulations that are suitable for various types of administration can be found in Remington's Pharmaceutical Sciences, Mace Publishing Company, Philadelphia, Pa., 17th ed. (1985) and corresponding updates. For a brief review of methods for drug delivery see Langer, Science 249 (1990), 1527-1533.

The formulation may be presented in unit-dose or multi-dose containers, for example sealed ampoules and vials, or may be stored in a frozen or freeze-dried (lyophilized) condition requiring only the addition of sterile liquid carrier immediately prior to use. Preferably, the formulation is supplied as sterile suspension in a buffered physiological saline (0.9% w/v sodium chloride) aqueous solution in single-use vials. In a preferred embodiment, the pulmonary surfactant or biologically active derivative thereof is present in the formulation at a concentration from about 0.005 mg/ml to about 100 mg/ml, preferably from about 0.005 mg/ml to about 50 mg/ml, more preferably from about 0.01 mg/ml to about 5 mg,/ml, even more preferably from about 0.01 mg/ml to about 0.5 mg/ml. Since, as shown in the Examples, a concentration of 0.01 mg/ml of an exemplary modified pulmonary surfactant has shown the best enhancement of skin wound closure, in a particularly preferred embodiment the pulmonary surfactant or biologically active derivative thereof is present in the formulation at a concentration from about 0.01 mg/ml to about 0.1 mg/ml.

The volume of the formulation in which the pulmonary surfactant or biologically active derivative are suspended will depend on the desired concentration. Advantageously, the volume of the formulation should be not more than 5.0 ml, preferably from 4.0 to 0.5 ml, more preferably from 2.0 to 1.0 ml. The dosage regimen will be determined by the attending physician and clinical factors. As is well known in the medical arts, dosages for any one patient depends upon many factors, including the patient's or animal's size, body surface area to be treated age, the particular compound to be administered, sex, time and route of administration, general health, and other drugs being administered concurrently. As the areas treated with the pulmonary surfactant or biologically active derivative thereof are approximately planar, the doses are described in relation to surface in the following.

In a typical unit dose for the composition for use according to the present invention the pulmonary surfactant or biologically active derivative thereof is administered at a dosage from about 0.01 μg/mm² to about 100 mg/mm² or to about 10 mg/mm², preferably from about 0.05 μg/mm² to about 1 mg/mm², more preferably from about 0.05 μg/mm² to about 0.5 mg/mm², still more preferably from about 0.1 μg/mm² to about 25 μg/mm² or from about 0.1 μg/mm² to about 10 μg/mm², and most preferably from about 0.1 μg/mm² to about 2 μg/mm² or from about 0.1 μg/mm² to about 0.5 μg/mm², for example, at a dosage of about 0.2 μg/mm² as shown in the Examples for the advantageous unit dose of 0.01 mg/ml in the Examples; however, doses below or above this range are envisioned, especially considering the aforementioned factors.

Progress of the treatment or use can be monitored by periodic assessment. Preparations for topical, intralesional, intraepithelial, intra-epidermal, intra-cutaneous or sub-cutaneous administration on or into the skin include, e.g., aqueous or non-aqueous solutions, suspensions, and emulsions including amphiphilic (also known as ambiphilic) and/or lipophilic solvents and formulations. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Preservatives and other additives may also be present such as, for example, antimicrobials, anti-oxidants, chelating agents, and inert gases and the like. Furthermore, the pharmaceutical composition of the invention may comprise further agents, depending on the intended use of the pharmaceutical composition.

As illustrated in Example 2 in the standardized excisional wound healing model in mice wound closure could be achieved with bovactant in accordance with the present invention when once applied every other day. Accordingly, in one embodiment of the present invention the pulmonary surfactant or biologically active derivative thereof is administered once about every other day. In a particularly preferred embodiment, the composition for use according to the present invention is adapted or designed such that the pulmonary surfactant or biologically active derivative thereof is administered onto the skin at a dose from about 0.1 μg/mm² to about 1 mg/mm² once about every other day.

In addition to administration of the pulmonary surfactant or biologically active derivative thereof in accordance with the present invention, co-administration administration of other therapeutic agents may be desirable, for an antibiotic, a antimycotic or an analgesic, for preventing an infection and ameliorate pain, respectively, typically associated with skin disorders, in particular wounds. Accordingly, in an embodiment the composition for use according to the present invention further comprises a further active agent. Preferably, the further agent is selected from a skin-active agent and/or a therapeutic agent such as an anti-inflammatory drug, anti-fibrotic agent, pro-migratory agent, angiogenesis and wound healing enhancing agent, agent reducing scarring and skin irritation, anti-allergic, antibiotic, antimycotic or analgesic drug.

Alternatively, when the pulmonary surfactant or biologically active derivative thereof and the one or more further agents are administered separately, each individual active component may be formulated separately. In this case, the individual active components do not unconditionally have to be taken at the same time. In the case of a separate administration, the formulation of each individual active component may be packed at the same time in a suitable container to form a kit.

As illustrated in the Examples, the modified natural pulmonary surfactant is particularly effective in enhancing wound closure when applied onto skin. Accordingly, the present invention provides and relates to a pharmaceutical composition for use in the topical administration on the skin comprising any one of the above described compositions and formulations of a pulmonary surfactant or a derivative thereof. Thus, with respect to the treatment of disorders of the skin, the pulmonary surfactant or biologically active derivative thereof is preferably the sole therapeutically or cosmetically active ingredient in the composition for use in accordance with the present invention.

The composition or formulation of a pulmonary surfactant or a biologically active derivative for use in accordance with the present invention may be manufactured in a kit, pharmaceutical or cosmetic package, for example comprising a first container comprising the composition or formulation and a second container comprising for example a second to be applied in conjunction with the composition and formulation, respectively, a physiologically acceptable aqueous diluent, and instructions for topical administration on the skin.

In addition, the present invention relates to a kit or a pharmaceutical or cosmetic package comprising a first container comprising a pulmonary surfactant or a biologically active derivative thereof as defined herein, preferably in dried form and a second container comprising a physiologically acceptable aqueous diluent, and instructions for topical administration on the skin. Associated with the kits respective the packages of the present invention, e.g., within a container comprising the kit or package can be a notice in the form prescribed by a governmental agency regulating the manufacture, use or sale of pharmaceutical products, which notice reflects approval by the agency of manufacture, use or sale for human administration. In case the kit or package is designed for cosmetic use, it comprises instructions for appropriate use. Furthermore, the kit or package can contain means for mixing the pulmonary surfactant or a biologically active derivative thereof with the physiologically acceptable aqueous diluent, such as syringes, pipettes, spoons and/or means for administering the mixture to the skin, such as syringes, pipettes, plasters, gauzes, garments, polyester or polypropylene nets, after the mixture has been taken up, impregnated or incorporated in these. In a preferred embodiment of the kit of the present invention, the modified pulmonary surfactant, preferably bovactant is present in the container, for example vial in dried form and in a concentration between 25 and 100 mg, preferably 50 mg. In another preferred embodiment, the composition or formulation of the modified pulmonary surfactant, preferably bovactant is designed in the kit ready-to-use, for example as a skin patch.

As mentioned and illustrated in the Examples, the modified natural pulmonary surfactant is particularly effective in enhancing wound closure without excessive scarring and inducing an anti-inflammatory effect when applied onto skin. Accordingly, the present invention provides and relates to a skin wound care treatment, dermatological or cosmetic product comprising a composition of the pulmonary surfactant or a biologically active derivative thereof as defined herein, which is effective for bringing about an anti-inflammatory, pro-migratory, and/or in particular an anti-fibrotic and/or enhanced wound closure effect on the skin. Put in other words, the present invention provides and relates to a pulmonary surfactant for use as an anti-inflammatory medicament in the treatment of skin disorders described herein, preferably by topical administration onto the skin. Typically, skin medical and cosmetic care products are manufactured in the form of or implemented in a preferably inert carrier such as a patch, plaster, gauze, garment, polyester or polypropylene net, and the like; see, e.g. international application WO 2007/137881 which describes a wound care treatment product on the basis of honey. Thus, the product of the present invention can be of various forms, as these are known in respect of medical products in the field of wound treatment or in the field of cosmetics for skin care products. In a preferred embodiment the product according to the present invention is:
(a) a local applicable product such as a gel, an ointment, a lotion, or a cream;
(b) a carrier consisting of a plaster, a gauze, a garment, silicon, a polyester or a polypropylene net onto which the composition, the gel, ointment, lotion or cream is applied resulting in a skin patch, fatty gauze, pressure garment, silicon sheet, a hydrocellular (polyurethane) foam plate wherein the composition, gel, ointment, lotion or cream is impregnated or incorporated, or an alginate that is mixed with the composition, gel, ointment, lotion or creme;
(c) a spray;
(d) any other skin wound treatment product or healing therapy; or
(e) a product for use in a keratinocyte, skin and/or wound healing assay.

The last product includes but is not limited to kits and compositions optionally further comprising, for example, cells such as keratinocytes, cell culture media, cell culture plates, standardized concentrations of pulmonary surfactants and/or derivatives thereof, solvents which are particularly useful in assays as described in Examples 1 and 2, which may be further designed for, e.g., screening of skin active agents or toxicological testing.

These and other embodiments are disclosed and encompassed by the description and examples of the present invention. Further literature concerning any one of the materials, methods, uses and compounds to be employed in accordance with the present invention may be retrieved from public libraries and databases, using for example electronic devices. For example the public database “Medline” may be utilized, which is hosted by the National Center for Biotechnology Information and/or the National Library of Medicine at the National Institutes of Health. Further databases and web addresses, such as those of the European Bioinformatics Institute (EBI), which is part of the European Molecular Biology Laboratory (EMBL) are known to the person skilled in the art and can also be obtained using internet search engines. An overview of patent information in biotechnology and a survey of relevant sources of patent information useful for retrospective searching and for current awareness is given in Berks, TIBTECH 12 (1994), 352-364.

The above disclosure generally describes the present invention. Several documents are cited throughout the text of this specification. Full bibliographic citations may be found at the end of the specification immediately preceding the claims. The contents of all cited references (including literature references, issued patents, published patent applications as cited throughout this application, including the disclosure in the background section and manufacturer's specifications, instructions, etc.) are hereby expressly incorporated by reference; however, there is no admission that any document cited is indeed prior art as to the present invention.

A more complete understanding can be obtained by reference to the following specific examples which are provided herein for purposes of illustration only and are not intended to limit the scope of the invention.

### EXAMPLES

### Materials and Methods

### Lung Surfactant

Wound healing and contraction were performed in a bottom-up style, beginning on the cellular level and ending with in vivo experiments. Standard surfactant used for respiratory distress syndrome therapy in children was tested by topical application in vitro and in vivo.

Surfactants are used as standard therapy for reducing alveolar surface tension in preterm infants³³. Phospholipid films with surfactant proteins regulate the shape and activity of alveolar macrophages by controlling surface tension³⁴. The important feature of this treatment is the lipophilic nature of the surfactant film. Hitherto, moist wound healing is the standard treatment for epidermal wounds. Applying surfactants on skin wounds is an innovative, outrageous and unorthodox, yet simple way to address skin wound healing and contraction. Surfactants custom-made for skin and applied on skin wounds could reduce surface tension and facilitate keratinocyte contraction. Hitherto, no published data exist on cutaneous surfactant therapy.

For treatment of Infant Respiratory Distress Syndrome (IRDS), bovine lung surfactant is commercially available in Germany with the trade name Alveofact®. In cell culture experiments performed in connection with the present invention, dose response experiments were performed with 0.01 mg/ml, 0.1 mg/ml and 1 mg/ml Alveofact® added to culture media. For in vivo experiments, Alveofact® was dissolved according to manufacturer's instructions in 0.9% saline and added topically onto skin wounds at concentrations of 0.01 mg/ml and 0.5 mg/ml. Wound dressings were changed every other day with new application of Alveofact® or saline alone as control group.

### Experimental Setting

The effect of Alveofact on wound healing was analysed using standard experimental models including cell³⁵ and organ cultures³⁶ and in vivo excisional wound models in mice.³⁷

Analyses performed:
Wound margins were traced and wound area analysed.³⁷
Culture media and tissues were analysed for secretion and localization of proteins.
Histology, immunohistochemistry and immunofluorescence analyses³⁸
Biochemical and biomolecular analyses including substrate zymography,³⁹ Western immunoblotting,³⁹ ELISA, RT-PCR, gene array analysis⁴⁰.

### Gene Expression Analysis By Tailor-Made Array

Total RNA (mg) was isolated using the Aurum total fatty and fibrous tissue kit (Biorad, Munich, Germany). During reverse transcription, RNA was converted into fluorescein (Fl) and biotin (B) labelled cDNA. Specifically bound Fl and B labelled cDNAs were sequentially detected with a series of conjugate reporter, ultimately with Tyramide-Cy3 and Tyramide-Cy5. The hybridised chips were scanned six times with an Axon 400B_{—} scanner at different settings. Primary image analysis was performed using the software tool Gene Pix Pro 6.0.

### Statistics

The averaged values for each gene of gene expression analysis comprise nine replicates. Outliers amongst the gene replicates were eliminated according to the outlier test by Nalimov. An adaption of Student's t-test, e.g. Welch's t-test for unequal variances of the analysed samples was used for comparison of means. Values of p<0.05 were assumed as significant and expressed as Mean±SD (standard deviation) or SEM (standard error of the mean).

### Example 1

### Dose Response in Cell Culture

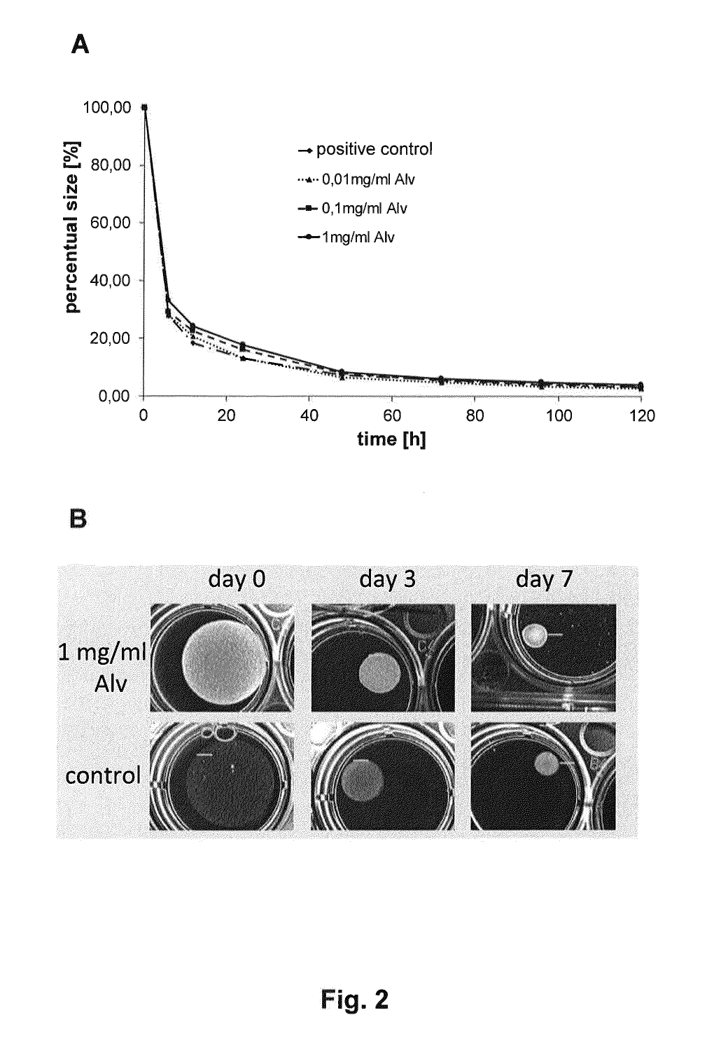

First, dose-response experiments were performed on keratinocyte monocultures using a scratch wounding model, wherein the cells (4× to 8×10⁴ cells per well) were suspended in culture medium either alone or including bovactant (Alveofact®). Alveofact® at 0.01 mg/ml promoted epithelial migration whereas concentration of 0.1 mg/ml had the same effect as control treatment and 1 mg/ml delayed keratinocyte migration (FIG. 1). Fibroblast migration and contractility was not affected by Alveofact® in low concentrations (FIG. 2 A, B).

### Example 2

### Excisional Wound Healing In Vivo

A standardized excisional wound healing model in mice was used to investigate cutaneous epithelial migration and wound contraction in mice in vivo. 8-mm punch biopsies were made on the back of mice and wounds closed with different dressings, i.e., 1 ml of 0.9% saline (control), 0.01 mg/ml or 0.5 mg/ml Alveofact® equivalent to a concentration of 0.2 μg/mm² and 10 μg/mm². As clinical gold standard for human wound treatment, fatty gauze was applied to wounds in a fourth group.

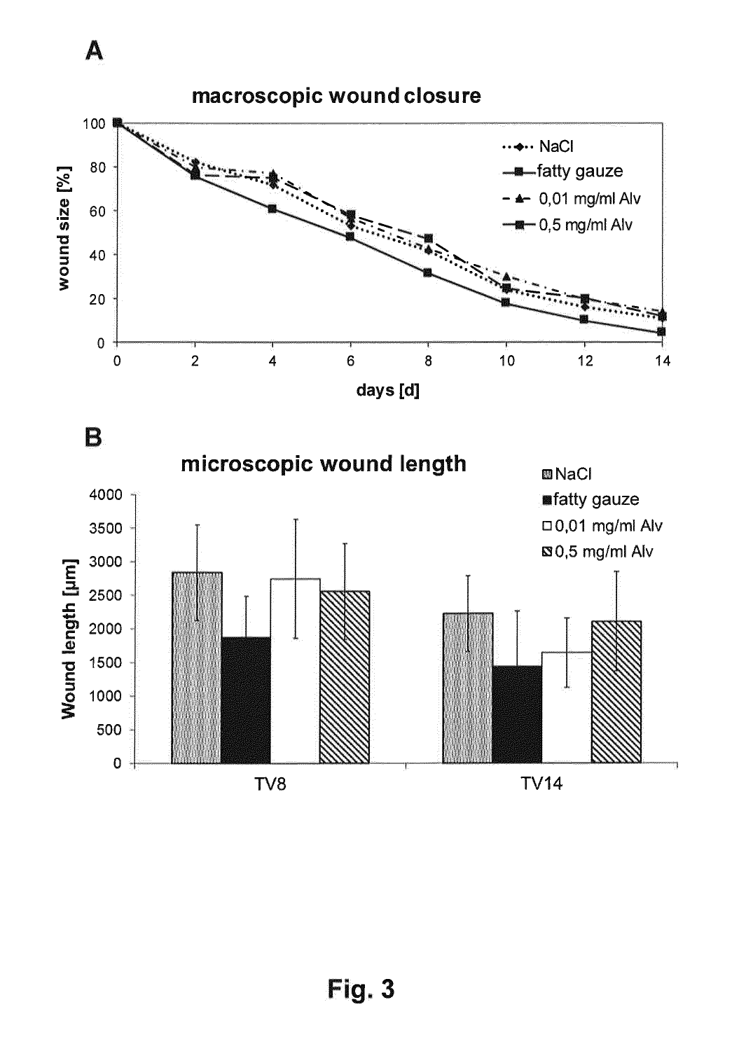

Fastest wound closure was observed with fatty gauze followed by Alveofact® at 0.01 mg/ml. The saline control paralleled wound closure rates of 0.5 mg/ml Alveofact®. This underlines results seen in cell culture experiments with Alveofact® 0.01 mg/ml being the optimal concentration for enhancement of skin wound closure (FIG. 3).

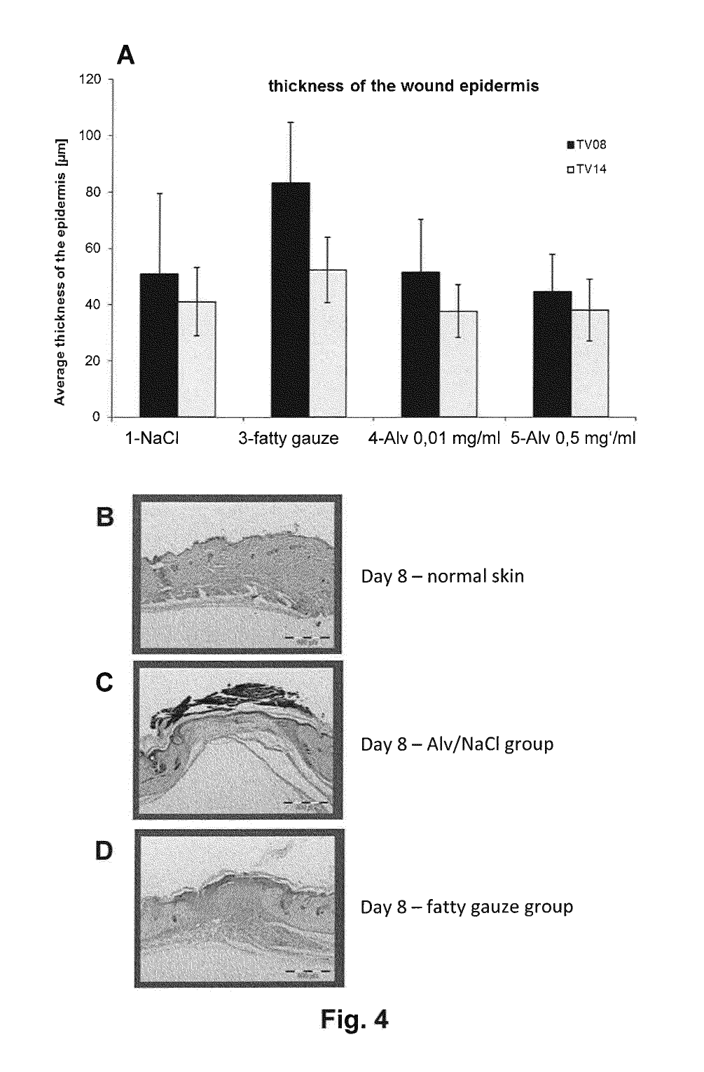

Histological analyses yielded highly interesting results. With fatty gauze treatment, skin width almost doubled in comparison with control or Alveofact® treatment and had the histological phenotype of a hypertrophic scar with cell rich and collagen rich dermal layer. With Alveofact® treatment, thin epidermal layers and also fluffy dermal compartment was noticed without any hint to excessive scarring (FIG. 4).

### Example 3

### Gene Expression Array Analysis

Wounds were snap frozen at −80 degrees centigrade and RNA extracted according to standard protocol. A tailor-made gene array was developed to analyse 159 genes involved for skin wound healing and cutaneous scarring. Subgroups of analysed genes according to family or function are stated in Annex I (Annex I. Genes expressed during wound healing analysed by gene array analysis). Gene expression of wounds treated with Alveofact® at different concentrations was compared to wounds treated with the vehicle (NaCL) alone at day 8 or day 14 postoperatively.

Significantly reduced values for proinflammatory TNF-alpha were found with Alveofact® 0.01 mg/ml and 0.5 mg/ml at day 8 and day 14. Alveofact® at 0.5 mg/ml reduced TACE expression significantly during the whole observation period. Furthermore, Alveofact® 0.01 mg/ml treatment reduced IL-1 beta expression significantly at day 14 (Tab. 1).

Downregulation of profibrotic TGF-beta2, TGFb-RI and MMP-3 were found at day 8 and, in part, at day 14. Significantly reduced values were found in both groups for the myofibroblast marker alpha-smooth-muscle actin (ASMA) and the AngiotensinII-Receptor-2 (ATII-R2) in comparison with control. Notably, profibrotic connective tissue growth factor (CTGF) was reduced with both Alveofact® concentrations at day 14 (Tab. 1).

Enhanced cellular migration observed in cell culture and microscopically during wound healing was reflected by elevation of promigratory genes in our gene array analysis during the whole observation period (Tab. 1). Promigratory MMP-13 expression was significantly increased with Alveofact treatment at day 8 and day 14 (Tab. 1).

### Gelatin Zymography and Western Immunoblotting

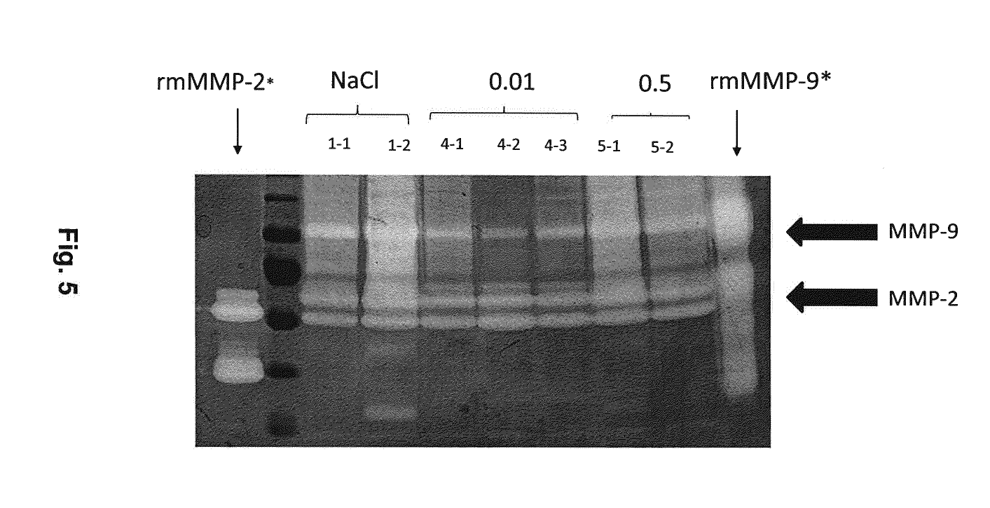

Reduced protein values for proinflammatory MMP-9 were found with Alveofact® 0.01 mg/ml compared to NaCl or Alveofact® 0.5 mg/ml (FIG. 5).

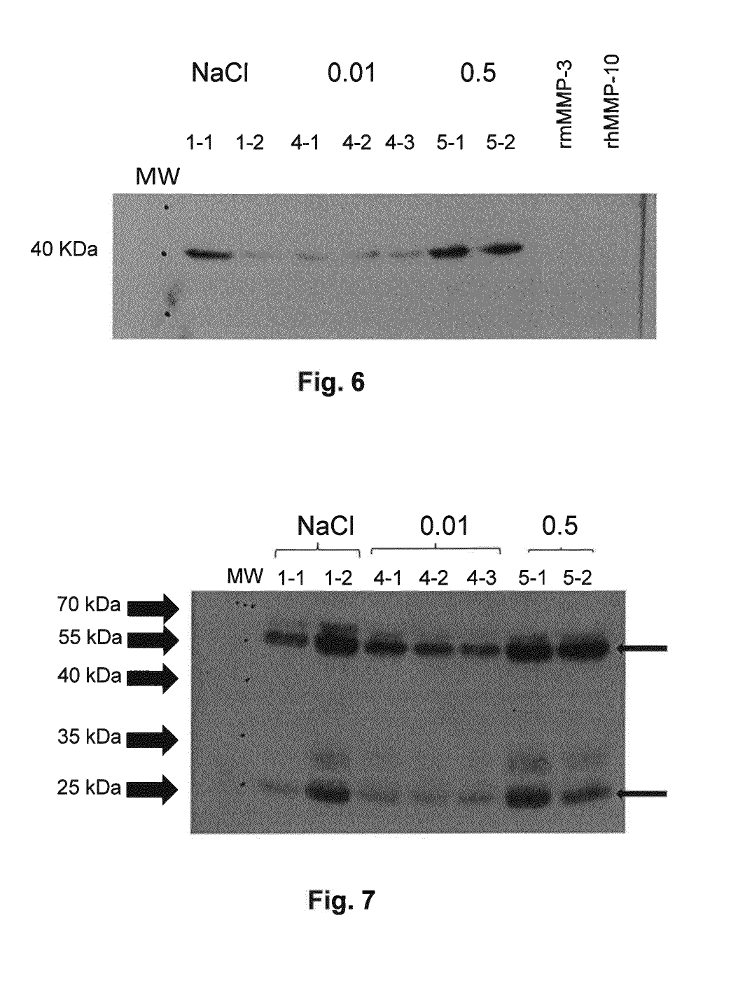

Western Immunoblotting for the myofibroblast marker ASMA showed reduced values with Alveofact® 0.01 mg/ml treatment in comparison to NaCl or Alveofact® 0.05 mg/ml (FIG. 6). E-cadherin is an intercellular contact protein of epithelial cells. During migration, E-cadherin levels are reduced as seen with Alveofact® 0.01 mg/ml treatment (FIG. 7).

### Conclusion

Lung surfactant and its components seem to have an anti-inflammatory, pro-migratory and anti-fibrotic effect on skin wound healing. This finding is novel and hitherto not described. Topical or intra-lesional application of lung surfactant or its components can have a beneficial effect on human or animal skin wound healing, e.g. acute, chronic or aberrant wound healing including scarring. By treatment of wounds with lung surfactant or its components, skin wound healing may be accelerated, local inflammation decreased and thereby wound closure enhanced and scar formation reduced. This would be an innovative and novel treatment strategy for skin wound healing and prevention for cutaneous scarring.

### REFERENCES

1 Ziegenthaler H, Neumann U, Fritzsche U et al. [Polytraumatized burn injury victims]. Orthopade 2005; 34: 906-16.
2 de Roche R. [Epidemiology]. In: Handbuch der Verbrennungstherapie (Bruck J C, Müller F E, Steen M, eds). Landsberg: Ecomed. 2002; 27-33.
3 German Burn Association DGV. Burn statistics. In, Vol. 2011: http://www.verbrennungsmedizin.de/statistik.php. 2009.
4 D'Souza A L, Nelson N G, McKenzie L B. Pediatric burn injuries treated in US emergency departments between 1990 and 2006. Pediatrics 2009; 124: 1424-30.
5 Anzarut A, Olson J, Singh P et al. The effectiveness of pressure garment therapy for the prevention of abnormal scarring after burn injury: a meta-analysis. J Plast Reconstr Aesthet Surg 2009; 62: 77-84.
6 Bloemen M C, van der Veer W M, Ulrich M M et al. Prevention and curative management of hypertrophic scar formation. Burns 2009; 35: 463-75.
7 Engrav L H, Heimbach D M, Rivara F P et al. 12-Year within-wound study of the effectiveness of custom pressure garment therapy. Burns 2010; 36: 975-83.
8 Stella M, Castagnoli C, Gangemi E N. Postburn scars: an update. Int J Low Extrem Wounds 2008; 7: 176-81.
9 van der Wal M B, Vloemans J F, Tuinebreijer W E et al. Outcome after burns: an observational study on burn scar maturation and predictors for severe scarring. Wound Repair Regen 2012; 20: 676-87.
10 Mirastschijski U, Sander J T, Weyand B et al. Rehabilitation of burn patients: an underestimated socio-economic burden. Burns 2013; 39: 262-8.
11 Mirastschij ski U, Sander J-T, Zier U et al. Post-burn scarring and treatment costs for patients of a German Burn Centre. In. Ann Burns Fire Disasters. submitted; 1-19.
12 Martin P. Wound healing-aiming for perfect skin regeneration. Science 1997; 276: 75-81.
13 Muller E J, Williamson L, Kolly C et al. Outside-in signaling through integrins and cadherins: a central mechanism to control epidermal growth and differentiation? The Journal of investigative dermatology 2008; 128: 501-16.
14 Tomasek J J, Gabbiani G, Hinz B et al. Myofibroblasts and mechano-regulation of connective tissue remodelling. Nat Rev Mol Cell Biol 2002; 3: 349-63.
15 Mirastschijski U, Jokuszies A, Vogt P M. Skin wound healing: repair biology, wound and scar treatment. In: Plastic Surgery (Neligan P C, ed), Vol. 1: Elsevier Saunders. 2012; 267-96.
16 Blakytny R, Jude E B. Altered molecular mechanisms of diabetic foot ulcers. Int J Low Extrem Wounds 2009; 8: 95-104.
17 Mishima Y, Kuyama A, Tada A et al. Relationship between serum tumor necrosis factor-alpha and insulin resistance in obese men with Type 2 diabetes mellitus. Diabetes Res Clin Pract 2001; 52: 119-23.
18 Loots M A, Lamme E N, Zeegelaar J et al. Differences in cellular infiltrate and extracellular matrix of chronic diabetic and venous ulcers versus acute wounds. The Journal of investigative dermatology 1998; 111: 850-7.
19 Nwomeh B C, Liang H X, Cohen I K et al. MMP-8 is the predominant collagenase in healing wounds and nonhealing ulcers. J Surg Res 1999; 81: 189-95.
20 Mirastschijski U, Impala U, Jahkola T et al. Ectopic localization of matrix metalloproteinase-9 in chronic cutaneous wounds. Human pathology 2002; 33: 355-64.
21 Acosta J B, del Barco D G, Vera D C et al. The pro-inflammatory environment in recalcitrant diabetic foot wounds. Int Wound J 2008; 5: 530-9.
22 Darby I A, Bisucci T, Hewitson T D et al. Apoptosis is increased in a model of diabetes-impaired wound healing in genetically diabetic mice. The international journal of biochemistry & cell biology 1997; 29: 191-200.
23 Sibbald R G, Orsted H, Schultz G S et al. Preparing the wound bed 2003: focus on infection and inflammation. Ostomy Wound Manage 2003; 49: 23-51.
24 Schultz G S, Sibbald R G, Falanga V et al. Wound bed preparation: a systematic approach to wound management. Wound Repair Regen 2003; 11 Suppl 1: S1-28.
25 Wassermann R J, Polo M, Smith P et al. Differential production of apoptosis-modulating proteins in patients with hypertrophic burn scar. J Surg Res 1998; 75: 74-80.
26 Colwell A S, Krummel T M, Longaker M T et al. Wnt-4 expression is increased in fibroblasts after TGF-betal stimulation and during fetal and postnatal wound repair. Plast Reconstr Surg 2006; 117: 2297-301.
27 Brigstock D R. Regulation of angiogenesis and endothelial cell function by connective tissue growth factor (CTGF) and cysteine-rich 61 (CYR61). Angiogenesis 2002; 5: 153-65.
28 Yamaguchi Y, Mann D M, Ruoslahti E. Negative regulation of transforming growth factor-beta by the proteoglycan decorin. Nature 1990; 346: 281-4.
29 Zhang Z, Li X J, Liu Y et al. Recombinant human decorin inhibits cell proliferation and downregulates TGF-beta1 production in hypertrophic scar fibroblasts. Burns 2007; 33: 634-41.
30 Scott P G, Dodd C M, Ghahary A et al. Fibroblasts from post-burn hypertrophic scar tissue synthesize less decorin than normal dermal fibroblasts. Clin Sci (Lond) 1998; 94: 541-7.
31 Mustoe T A, Cooter R D, Gold M H et al. International clinical recommendations on scar management. Plast Reconstr Surg 2002; 110: 560-71.
32 Liew S H, Murison M, Dickson W A. Prophylactic treatment of deep dermal burn scar to prevent hypertrophic scarring using the pulsed dye laser: a preliminary study. Ann Plast Surg 2002; 49: 472-5.
33 Halliday H L. Surfactants: past, present and future. J Perinatal 2008; 28 Suppl 1: S47-56.
34 Akei H, Whitsett J A, Buroker M et al. Surface tension influences cell shape and phagocytosis in alveolar macrophages. Am J Physiol Lung Cell Mol Physiol 2006; 291: L572-9.
35 Rehders M, Grosshauser B B, Smarandache A et al. Effects of lunar and mars dust simulants on HaCat keratinocytes and CHO-K1 fibroblasts. Advances in Space Research 2011; 47: 1200-13.
36 Mirastschijski U, Bugdahl R, Rollman O et al. Epithelial regeneration from bioengineered skin explants in culture. Br J Dermatol 2006; 154: 42-9.
37 Mirastschijski U, Haaksma C J, Tomasek J J et al. Matrix metalloproteinase inhibitor GM 6001 attenuates keratinocyte migration, contraction and myofibroblast formation in skin wounds. Exp Cell Res 2004; 299: 465-75.
38 Mirastschij ski U, Impala U, Jahkola T et al. Ectopic localization of matrix metalloproteinase-9 in chronic cutaneous wounds. Human pathology 2002; 33: 355-64.
39 Mirastschij ski U, Impola U, Karsdal M A et al. Matrix metalloproteinase inhibitor BB-3103 unlike the serine proteinase inhibitor aprotinin abrogates epidermal healing of human skin wounds ex vivo. The Journal of investigative dermatology 2002; 118: 55-64.
40 Aust M C, Reimers K, Kaplan H M et al. Percutaneous collagen induction-Regeneration in place of cicatrisation? J Plast Reconstr Aesthet Surg 2010; 64: 97-107.
41 Steinhusen U, Weiske J, Badock V et al. Cleavage and shedding of E-cadherin after induction of apoptosis. The Journal of biological chemistry 2001; 276: 4972-80.
42 Rüdiger M., Tölle A., Meier W. and Rüstow B. Naturally derived commercial surfactants differ in composition of surfactant lipids and in surface viscosity. Am J Physiol Lung Cell Mol Physiol 2005; 288: L379-L383.
43 Wright J R. Host defense functions of pulmonary surfactant. Biol Neonate. 2004; 85: 326-32.
44 Herting E, Rauprich P, Stichtenoth G et al. Resistance of different surfactant preparations to inactivation by meconium. Pediatr Res. 2001; 50:44-9.
45 Bernhard W, Mottaghian J, Gebert A, Rau G A, von Der HARDT H, Poets C F. Commercial versus native surfactants. Surface activity, molecular components, and the effect of calcium. Am J Respir Crit Care Med. 2000 October; 162(4 Pt 1):1524-33.
46 Hallman M, Teramo K, Ylikorkala O, Merritt T A. Natural surfactant substitution in respiratory distress syndrome. J Perinat Med. 1987;15(5):463-8.

1-20. (canceled)

21. A composition for use in the prevention or treatment of a disorder of the skin comprising a pulmonary surfactant, or a biologically active derivative thereof, and a pharmaceutically acceptable carrier, formulated for topical administration to the skin.

22. The composition of claim 21, wherein the pulmonary surfactant or biologically active derivative thereof has an anti-inflammatory effect on the skin.

23. The composition of claim 21, wherein the pulmonary surfactant is selected from the group consisting of poractant alfa, calfactant, bovactant, and beractant.

24. The composition of claim 21, wherein the pulmonary surfactant is bovactant.

25. The composition of claim 21, wherein the pharmaceutically acceptable carrier comprises a solvent.

26. The composition of claim 25, wherein the solvent is an aqueous, amphiphilic, or lipophilic solvent.

27. The composition of claim 21, wherein the pulmonary surfactant or biologically active derivative thereof is present in the formulation at a concentration from 0.005 mg/ml to 100 mg/ml.

28. The composition of claim 21, wherein the pulmonary surfactant or biologically active derivative thereof is present in the formulation at a concentration from about 0.01 mg/ml to about 0.5 mg/ml.

29. The composition of claim 21, wherein the pulmonary surfactant or biologically active derivative thereof is present in the formulation at a concentration from about 0.01 mg/ml to about 0.1 mg/ml.

30. The composition of claim 21, wherein the composition further comprises a further active agent selected from the group consisting of an anti-inflammatory agent, an anti-fibrotic agent, a pro-migratory agent, an angiogenesis enhancing agent, a wound healing agent, a scar reducing agent, a skin irritation reducing agent, an anti-allergic agent, and combinations thereof.

31. The composition of claim 21, wherein the composition is effective for bringing about an anti-inflammatory, pro-migratory, anti-fibrotic and/or enhanced wound closure effect on the skin.

32. The composition of claim 21, wherein the formulation is selected from:
(a) a topical gel, ointment, lotion, or cream;
(b) a skin patch, fatty gauze, pressure garment, silicon sheet, or hydrocellular foam plate impregnated with a gel, ointment, lotion, or cream; and,
(c) a topical spray.

33. A pharmaceutical package comprising a first container comprising a pulmonary surfactant or biologically active derivative thereof, and a second container comprising a physiologically acceptable aqueous diluent, and instructions for topical administration on the skin.

34. The pharmaceutical package of claim 33, wherein the pulmonary surfactant or biologically active derivative thereof in the first container is in a dried form.

35. A method of treatment of skin disorders such as wounds, local inflammation and/or scar formation comprising administering to a subject in need thereof a pulmonary surfactant or a biologically active derivative thereof in an amount effective for bringing about an anti-inflammatory, pro-migratory, anti-fibrotic and/or enhanced wound closure effect on the skin of the subject.

36. The method of claim 35, wherein the pulmonary surfactant or biologically active derivative thereof is administered in the form of a composition comprising a pharmaceutically acceptable carrier, formulated for topical administration to the skin.

37. The method of claim 35, wherein the administering comprises topical administration onto the skin of the subject.

38. The method of claim 35, wherein the skin disorder or condition is selected from the group consisting of skin wounds, fibrosis, burns, tissue augmentation, tissue defects, inflammation, irritation, allergy, inherited or acquired skin diseases with concomitant inflammation, benign or malignant malformations, and scar formation.

39. The method of claim 35, wherein the administering is conducted in combination with skin grafts, reconstructive surgery, dermatosurgery, or any topical, intra-epidermal or intra-cutaneous skin treatment.

40. The method of claim 35, wherein administering promotes epithelial migration of keratinocytes in a cosmetic treatment of the skin selected from the treatment of scars, wrinkles, discolorations of the skin, skin irritation, volume augmentation, baldness, after skin peeling, dermabrasion, and medical needling.
